(19) United States
(12) Patent Application Publication
(10) Pub. No.: US 2024/0398613 A1
(43) Pub. Date: Dec. 5, 2024

(54) SYSTEM OF COLD PACK BAGS PLACED ON THE SPINE AFTER INJURY TO MINIMIZE SWELLING

(71) Applicant: Joel A. Lafayette, Sonora, CA (US)

(72) Inventor: Joel A. Lafayette, Sonora, CA (US)

(21) Appl. No.: 18/674,270

(22) Filed: May 24, 2024

Related U.S. Application Data
(60) Provisional application No. 63/469,868, filed on May 31, 2023.

(51) Int. Cl. A61F 7/02; A61F 7/00

(52) U.S. Cl. CPC A61F 7/02; A61F 2007/0011; A61F 2007/023; A61F 2007/0279; A61F 2007/0282

(57) ABSTRACT

A system and method for treating spinal injuries comprising one or more cold pack bags placed on the spine after injury to minimize swelling. By adding cold packs to the spinal packaging process of spinal immobilization, the victim gains valuable time needed to get to a trauma center before swelling can cause secondary permanent damage.

### CROSS-REFERENCE TO RELATED APPLICATION

This application claims the benefit of priority of U.S. provisional application No. 63/469,868, filed May 31, 2023, the contents of which are herein incorporated by reference.

### BACKGROUND OF THE INVENTION

The present invention relates to the treatment of spinal injuries. Conventional protocol currently is to simply immobilize the victim with no cold pack application. The present invention is directed at improvements to the currently available protocols.

As is known by those skilled in the art, lower temperatures can promote healing and recovery by reducing inflammation and swelling. Currently, cold therapy for spinal cord injury involves an injection of saline to the areas around the spinal cord. This is commonly performed right after decompression surgery. The goal is to minimize secondary damage.

As is known by those skilled in the art, primary damage is the result of a traumatic event. Secondary damage develops as a result of the body's inflammatory response after injury. The present invention is directed at preventing and/or reducing the secondary damage with cold therapy applied in the field by First Responders through the use of cold packs.

### SUMMARY OF THE INVENTION

In one aspect of the present invention, the present invention helps prevent further injury to the spinal column after an initial traumatic event by adding cold packs to the spinal packaging process of spinal immobilization. By doing so, the victim gains valuable time needed to get to a trauma center before swelling can cause secondary permanent damage.

These and other features, aspects and advantages of the present invention will become better understood with reference to the following drawings, description, and claims.

### BRIEF DESCRIPTION OF THE DRAWINGS

FIG. 1 is a perspective view of the invention, shown in use;

FIG. 2 is an exploded view of the invention;

FIG. 3 is a section view taken along 3-3 in FIG. 1;

FIG. 4 is a section view showing motion; and

FIG. 5 is a perspective view of an alternate embodiment.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a product and a process to aid in preventing further injury to the spinal column after an initial traumatic event. The invention claimed here solves this and other problems associated with current conventional treatment products and processes.

In a preferred embodiment, the present invention cools the tissues of the injured area with cold medical packs, thus reducing swelling, and further protecting the spinal tissues.

Current protocol can actually cause additional swelling and secondary injury. In contrast, by adding cold packs in accordance with a preferred embodiment of the present invention to the spinal packaging process of spinal immobilization, the victim gains valuable time needed to get to a trauma center before swelling can cause secondary permanent damage.

Cold therapy helps minimize secondary damage by:
1. Slowing down the metabolism. Cold helps slow down body processes, which preserves oxygen and energy supplies.
2. Decreasing the production of free radicals, which cause cell damage through a process called oxidative stress.
3. Reducing excitotoxicity. Another secondary effect of spinal cord injury is the excessive release of neurotransmitters, which can overstimulate neurons and cause damage through the process of excitotoxicity.
4. Altering the permeability of the blood spinal cord barrier. Spinal cord injuries can disrupt this barrier, allowing harmful chemicals to enter the spinal cord.

Cold therapy slows the metabolism, decreases free radicals, reduces excitotoxicity, and helps preserve the blood spinal cord barrier to reduce inflammation and swelling. Additional benefits include:
Decreased bleeding;
Reduced muscle spasticity;
Improved motor function of affected limbs; and
Reduced pain.

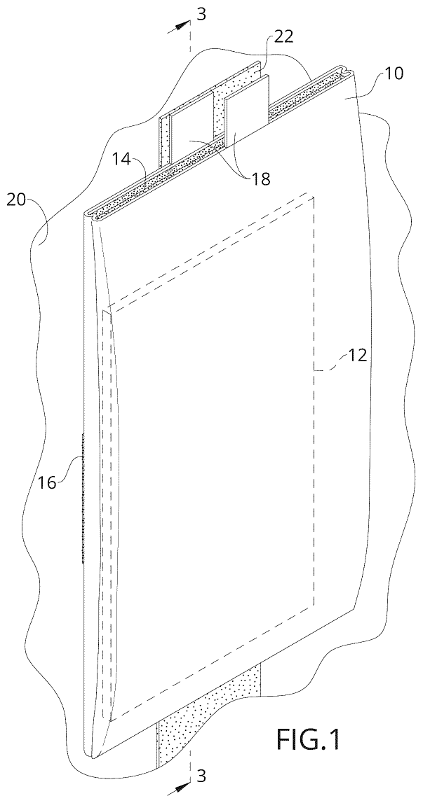

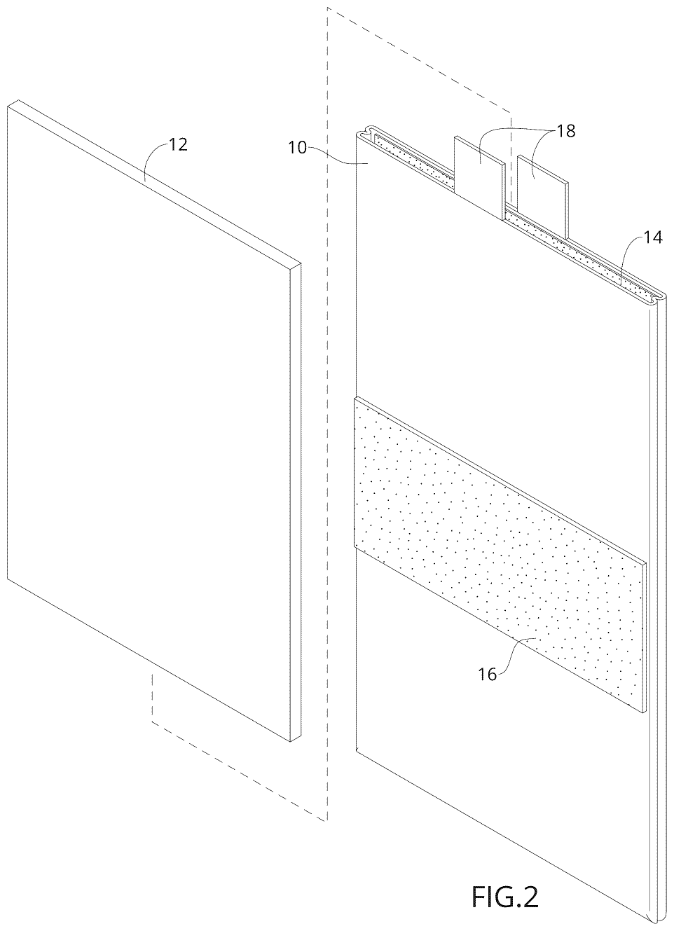

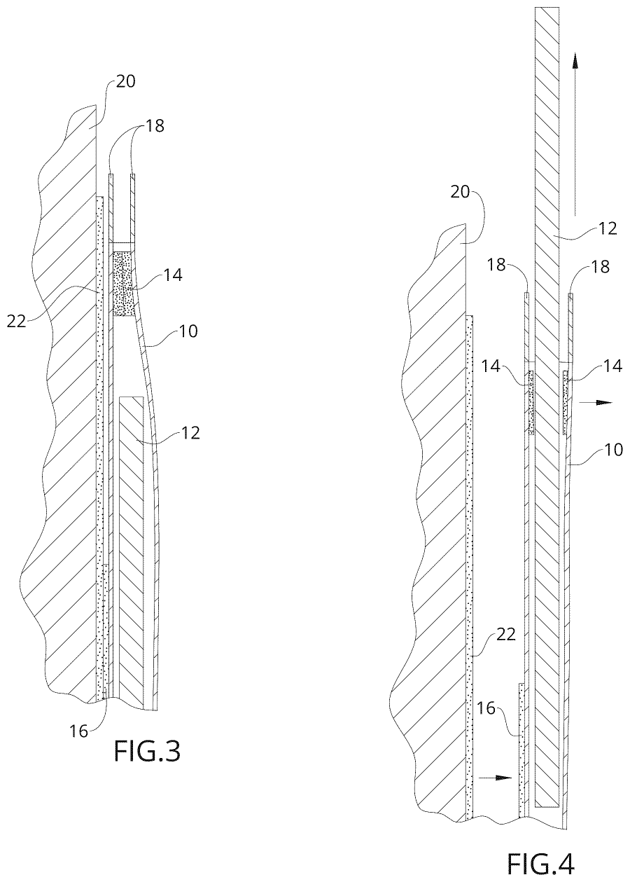

As described herein, and as seen by way of example in FIGS. 1-5 hereto, the present invention comprises integrating a system of pouches to be filled with emergency medical cold packs. One in the neck collar (c-spine) and a secondary “spider strap” type belt of canvas pockets filled each with the same readily available medical instant cold packs, to be placed against the victim's spinal column before being strapped to the backboard for transport.

As seen in FIGS. 1-5, in a preferred embodiment:
10 is the pouch;
12 is the cold pack;
14 are the inner hook and loop fasteners;
16 are the outer hook and loop fasteners;
18 are the tabs;
20 is the injury stabilization device;
22 are the injury stabilization device hook and loop fasteners;
30 is the alternate embodiment;
34 are the alternate embodiment inner hook and loop fasteners;
36 are the alternate embodiment outer hook and loop fasteners; and
38 are the alternate embodiment tabs.

As best seen in FIGS. 1-4, the cold pack 12 is positioned inside the pouch 10, and the hook and loop fasteners 14 help maintain the cold pack 12 in its position, together with the outer hook and loop fasteners 16 and tabs 18. Pouch 10 is then positioned and/or maintained and/or affixed to stabilization device 20 as appropriate via injury stabilization device hook and loop fasteners 22.

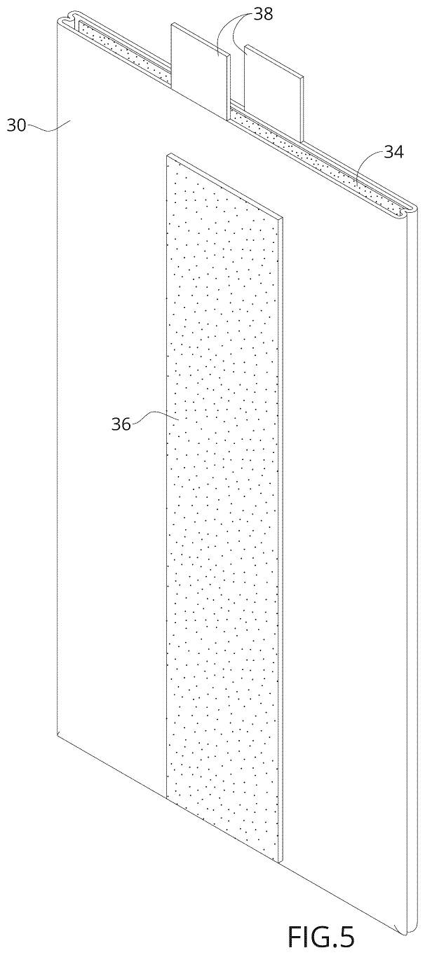

FIG. 5 depicts an alternative preferred embodiment, with inner hook and loop fasteners 34, outer hook and loop fasteners 36, and tabs 38.

In a preferred embodiment, a victim suffering a spinal injury would be treated in accordance with the present invention by following the following steps:
1. activate cold packs 12.
2. place a cervical collar as standard procedure dictates, with installed cold pack 12 in position at rear of collar in supplied strap pouch 10.
3. use the spinal strap of pouches in the same fashion (activate each cold pack 12) and strap onto victim with supplied Velcro/canvas/nylon pouch strap system similar to the common spider strap system.
4. transport the victim as normal.

The application of cold in a timely manner to spinal trauma has been shown to reduce or even eliminate secondary spinal damage caused by swelling. The “Back Buddy” as I call it (i.e., the present invention), is both a process and a product.

The process utilizes readily available cold packs 12 that EMT's and Paramedics already carry and use. My process indicates their use and placement whenever there is spinal trauma indicated, and the product (Back Buddy) simply holds these cold packs in place during transport.

This is accomplished through two separate web style strap systems. One single pouch strap for the neck collar, and a second strap system for the full back of a victim with sufficient length to cover all possible victim sizes. Fully adjustable and based on the same design platform as the current Spyder strap system used to attach victims to a back board for transport, the Back Buddy would be kept with the Spyder straps and backboard and used every time as standard procedure.

To make the present invention would comprise a simple web belt design with Velcro attachments to hold a canvas pouch or pouches of sufficient size to accommodate standard emergency cold packs. Web straps, Velcro, and canvas pouches are necessary in order to keep the cold packs in place during transport.

The pouches 10 are removable/replaceable. This allows for system expansion/reduction based on size of victim. It also allows for easier cleaning or replacement.

As described above, the present invention is directed towards the application of cold to prevent irreversible damage immediately after primary injury. In a preferred embodiment, treatment is typically performed in a manner that regulates the affected area to a temperature of 89.6-93.2 deg F. This allows for the benefits of the treatment to occur without the side effects associated with classic hypothermia.

In a preferred embodiment, cold therapy treatment should be applied as soon as possible to the area of suspected injury, and ideally continue for at least 3 or more hours, with a slow rewarming process for greater neuroprotection. The process I propose is simply a patient package and transport protocol that is designed to last approximately 30-45 minutes. This would cover the vast majority of transport times until the patient could be cared for at an advanced treatment facility. At that point, the decision could be made to use traditional saline injections or continue with external cold packs.

In a preferred embodiment, a two-inch webbing material can be used in connection with the present invention, such as Polyfab Shade Sail Edge Webbing (commonly used for shade sails and shade structures) which has a breaking strength of an approximate 4,700 pound force.

In a preferred embodiment, a one-inch webbing material can be used in connection with the present invention, which comprises a high-tenacity polyester webbing which resists mildew, UV rays, and shrinkage. The canvas straps are ideal for reinforcing swimming pool covers (both solid vinyl and screen). For smooth handling and sewing, the polyester webbing offers a low profile yet heavy weight material. Average Breaking 4800 lbs. strength:

In a preferred embodiment, the hook and loop fasteners can include VELCRO® Brand tape's lightweight, nylon assembly which provides durability, support, and effortless care, such as VELCRO® Brand nylon hook tape #194175 which has a width of 2 inches and is offered in black. It can be sewn on, or heat sealed.

In a preferred embodiment, Ottertex® Water-Repellent 200D (6.6 oz) DWR Polyester Ripstop fabric can be used in connection with the present invention which is a durable, crisp, smooth, and lightweight fabric.

It should be understood, of course, that the foregoing relates to exemplary embodiments of the invention and that modifications may be made without departing from the spirit and scope of the invention as set forth in the following claims.

What is claimed is:

1. A cold pack pouch for treating a spinal injury comprising:
a pouch comprising a front portion joined to a back portion and having an opening at one end to accept a cold pack,
the front portion and the back portion of the pouch comprising one or more tabs to aid in placing the cold pack within the opening of the pouch;
said pouch further comprising inner hook and loop fasteners to releasably close the opening in order to maintain the cold pack within the opening,
said pouch further comprising outer hook and loop fasteners on the front portion of the pouch to engage a corresponding hook and loop fastener on an injury stabilization device in order to maintain the cold pack pouch in an appropriate position to treat the spinal injury.

2. A method of treating a spinal injury of a victim with a cold pack pouch, said method comprising:
activating a cold pack;
placing the activated cold pack within an opening of a cold pack pouch;
closing the pouch to maintain the activated pack within the pouch;
placing a cervical collar on the victim with the activated cold pack in the pouch and positioned at the rear of the collar;
transporting the victim with the cold pack pouch in place to treat the spinal injury.
